(19) United States
(12) Patent Application Publication
(10) Pub. No.: US 2008/0247981 A1
(43) Pub. Date: Oct. 9, 2008

(54) Vinylpyrrolidone/Vinyl Acetate Copolymer, Process for Producing the Same, and Use of the Same

(75) Inventors: Daisuke Imai, Osaka (JP); Keiko Izumi, Osaka (JP); Yoshitomo Nakata, Hyogo (JP)

Correspondence Address: CONNOLLY BOVE LODGE & HUTZ LLP, 1875 EYE STREET, N.W., SUITE 1100, WASHINGTON, DC, 20036, US

(73) Assignee: NIPPON SHOKUBAI CO., LTD., Osaka (JP)

(21) Appl. No.: 11/718,755

(22) PCT Filed: Nov. 4, 2005
(86) PCT No.: PCT/JP2005/020312

§ 371 Date: Feb. 8, 2008

(30) Foreign Application Priority Data
Nov. 5, 2004 (JP) 2004-322785

(51) Int. Cl. C08F 26/08; A61K 8/81; A61Q 5/06

(52) U.S. Cl. 424/70.15; 526/264

(57) ABSTRACT

To provide a vinylpyrrolidone/vinyl acetate copolymer which is extremely reduced in thermal yellowing and is usable even at high temperatures and which can dissolve in water to give a transparent solution. The vinylpyrrolidone/vinyl acetate copolymer of the present invention has a residual vinylpyrrolidone content of 100 ppm or less relative to a solid content of the copolymer. A 5% by weight aqueous solution of the copolymer has an NTU value of 4.0 or less. A 50% aqueous solution of the copolymer has a Hazen color number (APHA) according to JIS-K3331 of 350 or less, the Hazen color number being measured by the following accelerated test.

### TECHNICAL FIELD

The present invention relates to a vinylpyrrolidone/vinyl acetate copolymer useful as a raw material of a styling gel and the like, a process for producing such a copolymer, and a use of such a copolymer.

### BACKGROUND ART

A vinylpyrrolidone/vinyl acetate copolymer has been known to be useful as a raw material polymer in various cosmetics applications and the like, and has been used as one of the most suitable raw materials of a styling gel, particularly. More specifically, a vinylpyrrolidone/vinyl acetate copolymer which has a relatively low K value according to Fikentscher method (hereinafter, also referred to as simply “K value”) of about 50 or less and dissolves in water to give a transparent solution, is preferable as the vinylpyrrolidone/vinyl acetate copolymer used in various cosmetics applications such as a styling gel. Such a vinylpyrrolidone/vinyl acetate copolymer has been commonly used, generally.

A process for performing polymerization in an organic solvent in the presence of a radical initiator has been proposed as a production process of the above-mentioned vinylpyrrolidone/vinyl acetate copolymer which has a low K value and can dissolve in water to give a transparent solution. Specific examples of such a process include a process in which main polymerization of a monomer component except for part of vinylpyrrolidone is performed using an azo initiator and then the rest of the vinylpyrrolidone is added and post-polymerization is performed using a specific initiator (referring to Patent Document 1) and a process in which polymerization is performed until an amount of unreacted vinylpyrrolidone becomes within a specific range, and then an organic solvent and the like is removed by distillation and water is further added and post-polymerization is performed using another radical initiator (referring to Patent Document 2). In addition to these processes, a process of performing polymerization in an aqueous medium may be mentioned as the production process of the vinylpyrrolidone/vinyl acetate copolymer. However, such a production process is hard to adopt for obtaining a copolymer which can dissolve in water to give a transparent solution, because an aqueous solution of the copolymer obtained by such polymerization in an aqueous medium easily causes turbidity or coloring.

### [Patent Document 1]

Japanese Kokai Publication No. Hei-08-3228

### [Patent Document 2]

### Japanese Patent No. 2974747 Publication

### SUMMARY OF THE INVENTION

Colorless or light-colored products have recently become mainstream products in cosmetics in general including hair styling products, with change in customer preference. Therefore, if a conventionally-known vinylpyrrolidone/vinyl acetate copolymer is used as a raw material, there is a problem in that a product has a strong yellow color and a product with a desired color may not be obtained. This problem is considered to be caused because the vinylpyrrolidone/vinyl acetate copolymer is easily yellow-colored by heat. Such a problem is remarkably caused, if, in view of productivity, a styling gel, for example, is produced at high temperatures in order to suppress bubbles from generating when each component is mixed. Therefore, in order to obtain products with no yellow color when various cosmetics are produced using the vinylpyrrolidone/vinyl acetate copolymer as a raw material, the production must have been performed except at high temperatures without pursuing advantages obtained when the production is performed at high temperatures, such as productivity. Therefore, a vinylpyrrolidone/vinyl acetate copolymer having a reduced property causing yellow coloring by heat (thermal yellowing) is needed as a raw material which has an excellent film-forming property, forms a soft and lustered film, has high redissolution in water, and is usable at high temperatures. However, conventional vinyl pyrrolidone/vinyl acetate copolymers including those disclosed in Patent Documents 1 and 2 do not have sufficient thermal yellowing resistance.

Addition of a reducing agent and a chelating agent together with the polymer has been proposed as a measure for suppressing the thermal yellowing, in production of a styling gel. However, addition of other components such as a reducing agent and a chelating agent leads to increase in production costs and causes a problem of precipitation generated after prolonged use. Therefore, the problem to be solved by the present invention is to provide: a vinylpyrrolidone/vinyl acetate copolymer which has an extremely low thermal yellowing and is usable even at high temperatures and which can dissolve in water to give a transparent solution; a process for producing such a polymer, and a use of such a polymer.

The present inventor made various investigations for solving the above-mentioned problems. Then, the inventor noted that a vinylpyrrolidone/vinyl acetate copolymer has an excellent film-forming property, forms a soft and lustered film, and has a high redissolution in water, and therefore, if used as a styling gel, such a copolymer has advantages in that the copolymer has a high hairset retention, and provides elegant and smooth texture and beautiful appearance for hair, and can be easily removed by washing. The inventor found that a vinylpyrrolidone/vinyl acetate copolymer obtained by terminating polymerization when a ratio of a residual N-vinylpyrrolidone amount to a residual vinyl acetate amount becomes a specific ratio can be a transparent solution by dissolving in water and have an extremely low thermal yellowing, although conventionally the polymerization is performed for a very long time to consume N-vinylpyrrolidone as complete as possible, for example, by performing the polymerization by further adding a radical initiator after an unreacted N-vinylpyrrolidone amount becomes within a specific range, as the method in Patent Document 2. The inventor further found that if such a vinylpyrrolidone/vinyl acetate copolymer has a Hazen color number (APHA) of a specific value or less when measured for thermal yellowing by a specific accelerated test, such a copolymer can surely provide a product which is almost or completely free from yellow coloring even if used at high temperatures (used in production of various products, performed at high temperatures). As a result, the present invention has been completed.

That is, the vinylpyrrolidone/vinyl acetate copolymer of the present invention is a vinylpyrrolidone/vinyl acetate copolymer having a residual vinylpyrrolidone content of 100 ppm or less relative to a solid content of the copolymer, the copolymer in 5% by weight aqueous solution form having an NTU value of 4.0 or less, and the copolymer in 50% by weight aqueous solution form having a Hazen color number (APHA) according to JIS-K3331 of 350 or less, the Hazen color number being measured by the following accelerated test.

The vinylpyrrolidone/vinyl acetate copolymer satisfies all of the above-mentioned requirements.

Accelerated test: the aqueous solution 10 g is charged into a 20 mL cylindrical glass container with an inner diameter of 24 mm and a depth of 50 mm, and the container is sealed and kept inside a thermostatic device at 80° C. for 72 hours.

A styling gel of the present invention comprises the vinylpyrrolidone/vinyl acetate copolymer of the present invention.

The production process of the vinylpyrrolidone/vinyl acetate copolymer of the present invention is a process for producing the vinylpyrrolidone/vinyl acetate copolymer of the present invention, wherein polymerization of a monomer component comprising N-vinylpyrrolidone and vinyl acetate is performed in a solvent in the presence of a polymerization initiator, the solvent containing water and an alcohol at a ratio (ratio by weight) of water/alcohol of 1/9 to 6/4, and the polymerization is terminated when a ratio (ratio by weight) of residual N-vinylpyrrolidone/residual vinyl acetate is 1/10 or more, and the alcohol is removed by distillation until the alcohol accounts for 10% by weight or less in a solution of the copolymer.

The vinylpyrrolidone/vinyl acetate copolymer and the production process of such a copolymer, and a styling gel using such a copolymer or production process, according to the present invention, are mentioned below in more detail, but not limited to these explanations. It should be understood that the present invention is not limited to exemplary embodiments described below, and can be appropriately modified within a scope or spirit of the present invention.

### “Vinylpyrrolidone/Vinyl Acetate Copolymer”

The vinylpyrrolidone/vinyl acetate copolymer of the present invention has a residual vinylpyrrolidone content of 100 ppm or less relative to a solid content of the copolymer. Such a copolymer having a residual vinylpyrrolidone content of 100 ppm or less relative to a solid content of the copolymer can be preferably used as a raw material of a styling gel and the like in view of toxicity, smelling stability, and storage stability. If the residual vinylpyrrolidone content is more than 100 ppm, such a copolymer may not be preferably used as a raw material of a styling gel and the like. The residual vinylpyrrolidone content is preferably 70 ppm or less, and more preferably 50 ppm or less, and most preferably 20 ppm or less.

The residual vinylpyrrolidone can be measured by liquid chromatography under the following conditions.
Column: product of Shiseido Co., Ltd., CAPCELL PACC18 TYPE UG120
5 μm, 1.5 mm(×250 mm
Eluent: methanol/water= 1/24 (containing 0.4% of sodium
1-heptanesulfonate)
Flow rate: 100 μL/min
Column oven: 20° C.
Injection volume: 10 μL
UV detector: 235 nm

A 5% by weight aqueous solution of the vinylpyrrolidone/vinyl acetate copolymer of the present invention shows an NTU (Nephelometric Turbidity Unit) value of 4.0 or less. Such an aqueous solution showing the above-mentioned NTU value of 4.0 or less dissolves in water to give a transparent solution, and therefore can be preferably used as a raw material of a styling gel, for example. The NTU value is preferably measured according to JIS-K0400-9-10.

A 50% by weight aqueous solution of the vinylpyrrolidone/vinyl acetate copolymer of the present invention shows a Hazen color number (APHA) according to JIS-K3331 of 350 or less when subjected to the following accelerated test. Such a copolymer showing a Hazen color number (APHA) measured by a specific accelerated test of 350 or less can surely provide a product which is almost or completely free from yellow coloring even if used at high temperatures (used in production of various products, performed at high temperatures).

Accelerated test: the aqueous solution 10 g is charged into a 20 mL cylindrical glass container with an inner diameter of 24 mm and a depth of 50 mm, and the container is sealed and kept inside a thermostatic device at 80° C. for 72 hours.

The above-mentioned aqueous solution in the accelerated test has a residual solvent of 1% or less.

The vinylpyrrolidone/vinyl acetate copolymer of the present invention preferably has a K value according to Fikentscher method of 50 or less, and more preferably 45 or less. If the K value according to Fikentscher method is more than 50, use for the application capable of utilizing the low thermal yellowing, which is a characteristic of the vinylpyrrolidone/vinyl acetate copolymer of the present invention, is limited. Therefore, such a copolymer is not suitable as a raw material of a styling gel.

In the present invention, the K value is a value shown in a Finkentscher formula using values obtained by measuring a solution for viscosity at 25° C. with a capillary viscometer, the solution being prepared by dissolving the copolymer in any solvent at a concentration of 10% by weight or less. The Fikentscher formula is as follows.

(logηrel)/C=[(75Ko²)/(1+1.5KoC)]+Ko

K=1000Ko

C represents the number of grams of the vinylpyrrolidone/vinyl acetate copolymer in 100 mL of the solution. ηrel represents the viscosity of the solution to the solvent.

It is preferable that the vinylpyrrolidone/vinyl acetate copolymer of the present invention has a metal content of 10 ppm or less. Such a copolymer having a metal content of 10 ppm or less can be preferably used as a material for films formed using a resist pattern. If the metal content is more than 10 ppm, a defect maybe generated in the resist process. The metal content is more preferably 5 ppm or less, and most preferably 1 ppm or less.

It is preferable that the vinylpyrrolidone/vinyl acetate copolymer of the present invention contains an inorganic ionic compound such as sulfate, sulfite, and phosphate of 1000 ppm or less. If the content of the inorganic ionic compound is large, turbidity may be generated when such a copolymer is used in a styling gel and the like. The content of the inorganic ionic compound is more preferably 500 ppm or less, and still more preferably 100 ppm or less, and most preferably 50 ppm or less.

The vinylpyrrolidone/vinyl acetate copolymer of the present invention essentially contains a structural unit represented by the following formula (1), derived from N-vinylpyrrolidone and a structural unit represented by the following formula (2), derived from vinyl acetate. The vinylpyrrolidone/vinyl acetate copolymer of the present invention may contain a structural unit derived from a monomer copolymerizable with N-vinylpyrrolidone (another monomer), mentioned below in “production process of vinylpyrrolidone/vinyl acetate copolymer. The ratio of the structural unit derived from N-vinylpyrrolidone to the structural unit derived from vinyl acetate in the vinylpyrrolidone/vinyl acetate copolymer of the present invention is not especially limited. The structural unit derived from N-vinylpyrrolidone/the structural unit derived from vinyl acetate (ratio by weight) is preferably 4/6 to 9/1, and more preferably 5/5 to 8/2. If the copolymer contains also the structural unit derived from another monomer, the proportion of the structural unit derived from another monomer is preferably 15% by weight or less and more preferably 10% by weight or less relative to all of the structural units.

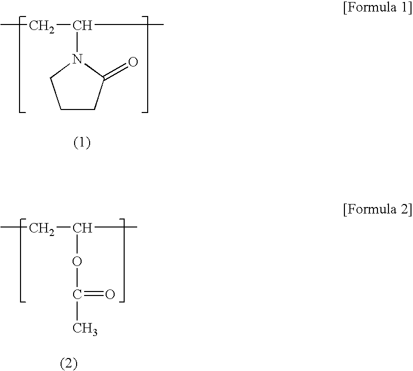

The vinylpyrrolidone/vinyl acetate copolymer of the present invention can be easily produced by the following process for producing the vinylpyrrolidone/vinyl acetate copolymer of the present invention, but is not limited to the process.

The application of the vinylpyrrolidone/vinyl acetate copolymer of the present invention is not limited and the copolymer can be used in various applications. Examples of the application in which the characteristics of the copolymer can be most effectively utilized include styling gels and adhesives. Among them, the copolymer is preferably used in styling gels.

### “Styling Gel”

The styling gel of the present invention includes the above-mentioned vinylpyrrolidone/vinyl acetate copolymer of the present invention. Specifically, the styling gel of the present invention includes the vinylpyrrolidone/vinyl acetate copolymer of the present invention as a main raw material copolymer, and can be easily obtained by mixing commonly used components such as a carboxyvinyl polymer, a surfactant, an oil-based substance, a polyalcohol, a gum substance, a natural water-soluble high molecular compound or a derivative thereof, an antiseptic, a chelating agent, an antioxidant, an ultraviolet absorber, a pigment, and a perfume, with the copolymer at high temperatures. The proportion of the above-mentioned vinylpyrrolidone/vinyl acetate copolymer in the styling gel of the present invention and the proportion of the other mixing components are not especially limited, and maybe appropriately determined within the proportion commonly employed in production of styling gels.

### “Production Process of Vinylpyrrolidone/Vinyl Acetate Copolymer”

The production process of the vinylpyrrolidone/vinyl acetate copolymer of the present invention is a process for producing the above-mentioned vinylpyrrolidone/vinyl acetate copolymer of the present invention. In the production process, a monomer component essentially containing N-vinylpyrrolidone and vinyl acetate is polymerized in a specific solvent in the presence of a polymerization initiator.

The above-mentioned monomer component essentially contains N-vinylpyrrolidone and vinyl acetate, and may further contain a monomer copolymerizable with N-vinylpyrrolidone (another monomer). The another monomer is not especially limited, and specific examples thereof include: (1) (meth)acrylates such as methyl (meth)acrylate, ethyl (meth)acrylate, butyl (meth)acrylate, cyclohexyl (meth)acrylate, and hydroxyethyl (meth)acrylate; (2) (meth)acrylamide derivatives such as (meth)acrylamide, N-monomethyl (meth) acrylamide, N-monoethyl (meth) acrylamide, and N,N-dimethyl (meth)acrylamide; (3) basic unsaturated monomers such as dimethylaminoethyl (meth)acrylate, dimethylaminoethyl (meth)acrylamide, vinylpyridine, and vinylimidazole; (4) vinylamides such as vinylformamide, vinylacetamide, and vinyloxazolidone; (5) carboxyl group-containing unsaturated monomers such as (meth)acrylic acid, itaconic acid, maleic acid, and fumaric acid; (6) unsaturated acid anhydride such as maleic anhydride and itaconic anhydride; (7) vinyl esters such as vinyl propionate; (8) vinyl ethylene carbonate and derivatives thereof; (9) styrene and derivatives thereof; (10) (meth) acrylic acid-2-ethyl sulfonate andderivatives thereof; (11) vinylsulfonic acid and derivatives thereof; (12) vinyl ethers such as methyl vinyl ether, ethyl vinyl ether, and butyl vinyl ether; and (13) olefins such as ethylene, propylene, octene, and butadiene. Among these monomers, the monomers in (1) to (8) are particularly preferable in view of copolymerization with N-vinylpyrrolidone. One or two or more different monomers may be used as the above-mentioned another monomer.

The proportion of each monomer in the above-mentioned monomer component is not especially limited. As for the ratio of the N-vinylpyrrolidone to the vinyl acetate, a ratio (ratio by weight) of N-vinylpyrrolidone/vinyl acetate is 4/6 to 9/1, and more preferably 5/5 to 8/2. If the above-mentioned monomer component also contains the above-mentioned another monomer, the content of the another monomer accounts for 15% by weight or less and more preferably 10% by weight or less relative to the whole monomer component. Azo polymerization initiators such as 2,2′-azobisisobutyronitrile, 2,2′-azobis(2-methylpropioneamidine)dihydrochloride, dimethyl-2,2′-azobisisobutyrate, 4,4′-azobis(4-cyanovaleric acid) are preferable in view of polymerization efficiency as the above-mentioned polymerization initiator, for example. Among these, dimethyl-2,2′-azobisisobutyrate is especially preferable. The polymerization initiator is not limited thereto, and used may be conventionally-known polymerization initiators, for example, radical polymerization initiators such as peroxides such as benzoyl peroxide and hydrogen peroxide; and cationic polymerization initiators such as boron trifluoride or a complex thereof, iron(II) chloride, diethylaluminum chloride, diethyl zinc, heteropolyacid, and activated clay. One or two or more polymerization initiators may be used.

The use amount of the above-mentioned polymerization initiator is not especially limited, but the polymerization initiator preferably accounts for 0.001 to 10% by weight, and more preferably 0.01 to 5% by weight, and most preferably 0.05 to 3% by weight relative to the monomer component. In the polymerization reaction, any chain transfer agent, cocatalyst, pH adjustor, buffer and the like may be appropriately used if necessary, in combination with the polymerization initiator.

In the production process of the vinylpyrrolidone/vinyl acetate copolymer of the present invention, it is important to use a mixed solvent containing water and an alcohol at a ratio (ratio by weight) of water/alcohol of 1/9 to 6/4 as the above-mentioned specific solvent. A mixed solvent containing water and an alcohol at a ratio (ratio by weight) of water/alcohol of 2/8 to 5/5 is preferable. If water is beyond the above-mentioned range (alcohol is lower) in the mixed solvent, the obtained copolymer generates turbidity when dissolves in water. If water is below the above-mentioned range (alcohol is higher) in the mixed solvent, the vinyl acetate is hard to polymerize and therefore a copolymer with a desired copolymerization ratio may not be obtained.

Examples of the above-mentioned alcohol include methyl alcohol, ethyl alcohol, diethylene glycol, and isopropyl alcohol. Among them, isopropyl alcohol is the most preferable for further exhibition of the effects of the invention. One or two or more different alcohols may be used as the above-mentioned alcohol.

The above-mentioned specific mixed solvent must contain water and an alcohol as main components, but may contain another organic solvent as long as the another organic solvent accounts for 10% by weight or less in the mixed solvent. Examples of the another organic solvent include alkylene glycol ether (acetates) such as propylene glycol monomethyl acetate and diethylene glycol monomethylether acetate; amides such as dimethylformamide and N-methylpyrrolidone; esters such as ethyl acetate, butyl acetate, and γ-butyrolactone; aliphatic hydrocarbons such as hexane and octane; alicyclic saturated hydrocarbons such as cyclohexane; alicyclic unsaturated hydrocarbons such as cyclohexene; aromatic hydrocarbons such as benzene, toluene, and xylene; ketones such as acetone and methyl ethyl ketone; halogenated hydrocarbons such as dichloroethane, chloroform and carbon tetrachloride; ethers such as diethyl ether, dioxane, and tetrahydrofuran; sulfonates such as dimethyl sulfoxide; carbonates such as dimethyl carbonate and diethyl carbonate; and alicyclic carbonates such as ethylene carbonate and propylene carbonate.

The use amount of the above-mentioned specific mixed solvent is not especially limited and may be appropriately determined in view of productivity. It is preferable that the specific mixed solvent is used in such an amount that the concentration of the monomer component is 30% by weight or more.

In the above-mentioned polymerization, the way of charging the monomer component, the polymerization initiator, the mixed solvent and the like is not especially limited. However, particularly for the monomer component, sequential addition is better than en bloc addition. Specifically, the sequential addition maybe continuous addition (for example, an embodiment is which dropwise addition is performed over a specific time) or may be intermittent addition (for example, an embodiment in which each raw material (monomer component, hydrogen peroxide, and ammonia) is added in some portions). Alternatively, the addition may be performed by a combination of the continuous addition and the intermittent addition. It is preferable that completion of charging of the vinyl acetate is earlier than that of the N-vinylpyrrolidone in the monomer component, in order to obtain a copolymer with a desired copolymer ratio.

The polymerization temperature (reaction temperature) in the above-mentioned polymerization is preferably 60 to 75° C., and more preferably 65 to 72° C.

In the production process of the vinylpyrrolidone/vinyl acetate copolymer of the present invention, it is important that the above-mentioned polymerization is terminated when a ratio (ratio by weight) of residual N-vinylpyrrolidone/residual vinyl acetate is 1/10 or more. It is preferable that the polymerization is terminated when the ratio (ratio by weight) of residual N-vinylpyrrolidone/residual vinyl acetate is 1/8 or more. If the polymerization is terminated when the ratio (ratio by weight) of residual N-vinylpyrrolidone/residual vinyl acetate is less than 1/10 or more, that is, the polymerization reaction is allowed to proceed excessively, the obtained copolymer generates turbidity when dissolves in water. The termination of the polymerization means that the solvent starts to be removed from the reaction solution by the after-mentioned distillation performed after the polymerization.

The above-mentioned ratio (ratio by weight) of residual N-vinylpyrrolidone/residual vinyl acetate can be measured, for example, by analyzing the reaction solution by gas chromatography, iodometry, and the like, during the polymerization.

In the production process of the vinylpyrrolidone/vinyl acetate copolymer of the present invention, it is preferable that the reaction solution obtained in the above-mentioned polymerization is subjected to distillation to remove the vinyl acetate and the solvents other than water in the mixed solvent therefrom. It is preferable in the above-mentioned distillation that water in the same or about double amount of the alcohol in the mixed solvent used in the polymerization is added.

In the production process of the vinylpyrrolidone/vinyl acetate copolymer of the present invention, the above-mentioned polymerization is terminated and then, the alcohol is removed by distillation until the alcohol accounts for 10% or less relative to the polymer solution. Thus, if the distillation is performed to remove the alcohol until the alcohol accounts for 10% or less, the residual vinylacetate is removed by azeotropy with the alcohol. In addition, the temperature of the polymer solution increases, and thereby the residual vinyl pyrrolidone can be reduced. If the alcohol accounts for more than 10%, the residual vinyl acetate and the residual vinylpyrrolidone may not be effectively reduced. The alcohol preferably accounts for 5% or less, and more preferably 3% or less, and most preferably 1% or less. In the distillation step, about 10 to 1000 ppm of an acid such as acetic acid may be added for acceleration of reduction in the residual vinylpyrrolidone.

### EFFECT OF THE INVENTION

The present invention can provide a vinylpyrrolidone/vinyl acetate copolymer which has an extremely low thermal yellowing and is usable even at high temperatures, and which dissolves in water to give a transparent solution. Such a vinylpyrrolidone/vinyl acetate copolymer can be used at high temperatures as a raw material of a styling gel, for example. According to the present invention, a styling gel which has an excellent film-forming property, forms a soft and lustered film, has a high redissolution in water, and is not yellow-colored, can be produced at high temperatures with excellent productivity.

### BEST MODE FOR CARRYING OUT THE INVENTION

The present invention is mentioned below in more detail with reference to Examples, but is not limited thereto. Hereinafter, the terms, “part(s) by weight” and “% by weight” are represented by simply “part(s)” and “%”, respectively, unless otherwise specified.

### EXAMPLE 1

Into a reaction container equipped with a condenser, a nitrogen inlet line, and a thermometer, 40 parts of a mixed solvent having a ratio (ratio by weight) of water and isopropyl alcohol of 5/5, 3 parts of N-vinylpyrrolidone, and 3 parts of vinyl acetate were charged. The atmosphere inside the reaction container was made to be nitrogen atmosphere by introduction of nitrogen under stirring. Then, the reaction container was heated with an oil bath until the inner temperature of the reaction container was 70° C. Thereto, an initiator solution A prepared by dissolving 0.03 parts of dimethyl-2,2′-azobisisobutylate in 0.5 parts of a mixed solvent having a water/isopropylalcohol (ratio by weight) of 5/5 was added. Thereto, a monomer solution A including 22.5 parts of N-vinylpyrrolidone and 17 parts of vinyl acetate, and an initiator solution B prepared by dissolving 0.38 parts of dimethyl-2,2′-azobisisobutylate in 7.2 parts of a mixed solvent having a ratio (ratio by weight) of water/isopropyl alcohol of 5/5, were each added dropwise over 4 hours. After completion of the dropwise addition, a monomer solution B including 4.5 parts of N-vinylpyrrolidone and 0.28 parts of water, and an initiator solution C prepared by dissolving 0.01 parts of dimethyl-2,2′-azobisisobutylate in 1.8 parts of the mixed solvent having a water/isopropyl alcohol (ratio by weight) of 5/5, were each added dropwise over 1 hour. After completion of the dropwise addition, the polymerization reaction was performed under stirring for 45 minutes while the inner temperature was kept at 70° C. And immediately, 30 parts of water was added to the reaction solution to form a homogeneous solution. Then, a distillation device was put on the above-mentioned reaction container and the oil bath temperature was raised to 130° C., and then distillation was started. The residual N-vinylpyrrolidone/residual vinyl acetate (ratio by weight) was 1.7/10 when the solvent started to be removed from the reaction solution. After the removed amount reached 30 parts, an operation of adding 6.7 parts of water into the reaction container and removing 6.7 parts was repeated twice. Further, 0.005 parts of acetic acid and 6.7 parts of water were added and 6.7 parts was removed. Then, the reaction solution was cooled (at this point, the N-vinylpyrrolidone concentration in the reaction solution reduced from 1177 ppm that was a concentration before the distillation to 10 ppm or less; the vinyl acetate concentration in the reaction solution reduced from 7116 ppm that was a concentration before the distillation to 50 ppm or less; and the isopropyl alcohol accounted for 0.1% or less). As a result, a 51.5% transparent and colorless solution of a vinylpyrrolidone/vinyl acetate copolymer was obtained.

The obtained polymer solution had a K value of 26. A 50% solution of the copolymer had a Hazen color number (APHA) according to JIS-K3331 of 30, and a 5% solution of the copolymer had an NTU value according to JIS-K0400-9-10 of 0.4. Further, a 50% aqueous solution of the copolymer was prepared from the obtained polymer solution. The 50% aqueous solution 10 g was charged into a 20 mL cylindrical glass container with an inner diameter of 24 mm and a depth of 50 mm, and the container was sealed. The container was kept inside a thermostatic device at 80° C. for 72 hours, and thereby the solution was subjected to the accelerated test. The solution had a Hazen color number (APHA) according to JIS-K3331 of 260.

### EXAMPLE 2

Polymerization and distillation were performed in the same manner as in Example 1 to produce a 51.7% transparent and colorless solution of a vinylpyrrolidone/vinyl acetate copolymer, except that a mixed solvent having a ratio (ratio by weight) of water/isopropyl alcohol of 3/7 was used instead of the mixed solvent having a ratio (ratio by weight) of water/isopropyl alcohol of 5/5. In the distillation, the residual N-vinylpyrrolidone/residual vinylacetate (ratio by weight) was 1.9/10 when the solvent started to be removed from the reaction solution. At completion of the distillation (at the time of cooling of the reaction solution), the N-vinylpyrrolidone concentration in the reaction solution reduced from 3720 ppm that was a concentration before distillation to 10 ppm or less, and the vinyl acetate concentration in the reaction solution reduced from 19268 ppm that was a concentration before distillation to 50 ppm or less. The isopropyl alcohol accounted for 0.1% or less.

The obtained polymer solution had a K value of 26. A 50% solution of the copolymer had a Hazen color number (APHA) according to JIS-K3331 of 30, and a 5% solution of the copolymer had an NTU value according to JIS-K0400-9-10 of 0.7. Further, a 50% aqueous solution of the copolymer was prepared from the obtained polymer solution. The 50% aqueous solution 10 g was charged into a 20 mL cylindrical glass container with an inner diameter of 24 mm and a depth of 50 mm, and the container was sealed. The container was kept inside a thermostatic device at 80° C. for 72 hours, and thereby the solution was subjected to the accelerated test. The solution had a Hazen color number (APHA) according to JIS-K3331 of 220.

### EXAMPLE 3

Into a polymerization container equipped with a condenser, a nitrogen inlet line, and a thermometer, 40 parts of a mixed solvent having a ratio of water/isopropyl alcohol (ratio by weight) of 5/5, 3.9 parts of N-vinylpyrrolidone, and 2.6 parts of vinyl acetate were charged. The atmosphere inside the reaction container was made to be nitrogen atmosphere by introduction of nitrogen under stirring. Then, the polymerization container was heated with an oil bath until the inner temperature of the container was 70° C. Thereto, an initiator solution A prepared by dissolving 0.02 parts of dimethyl-2,2′-azobisisobutylate in 0.5 parts of isopropyl alcohol was added. Thereto, a monomer solution A including 29.3 parts of N-vinylpyrrolidone and 23.5 parts of vinyl acetate, and an initiator solution B prepared by dissolving 0.25 parts of dimethyl-2,2′-azobisisobutylate in 8 parts of isopropyl alcohol, were each added dropwise over 4 hours. After completion of the dropwise addition, a monomer solution B including 5.9 parts of N-vinylpyrrolidone and 12.6 parts of ion-exchanged water, and an initiator solution C prepared by dissolving 0.06 parts of dimethyl-2,2′-azobisisobutylate in 2.0 parts of isopropyl alcohol, were each added dropwise over 1 hour. After completion of the dropwise addition, the polymerization reaction was performed under stirring for 45 minutes while the inner temperature was kept at 70° C. And immediately, 30 parts of water was added to the reaction solution to forma homogeneous solution. Then, a distillation device was put on the above-mentioned reaction container and the oil bath temperature was raised to 130° C., and then distillation was started. The residual N-vinylpyrrolidone/residual vinyl acetate (ratio by weight) was 2.2/10 when the solvent started to be removed from the reaction solution. After the removed amount reached 30 parts, an operation of adding 6.7 parts of water into the reaction container and removing 6.7 parts was repeated three times. Then, the reaction solution was cooled (at this point, the N-vinylpyrrolidone concentration in the reaction solution reduced from 2659 ppm that was a concentration before the distillation to 23 ppm or less; the vinyl acetate concentration in the reaction solution reduced from 12138 ppm that was a concentration before the distillation to 50 ppm or less; and the isopropyl alcohol accounted for 0.1% or less). As a result, a 51.1% transparent and colorless solution of a vinylpyrrolidone/vinyl acetate copolymer was obtained.

The obtained polymer solution had a K value of 31. A 50% solution of the copolymer had a Hazen color number (APHA) according to JIS-K3331 of 30, and a 5% solution of the copolymer had an NTU value according to JIS-K0400-9-10 of 0.8. Further, a 50% aqueous solution of the copolymer was prepared from the obtained polymer solution. The 50% aqueous solution 10 g was charged into a 20 mL cylindrical glass container with an inner diameter of 24 mm and a depth of 50 mm, and the container was sealed. The container was kept inside a thermostatic device at 80° C. for 72 hours, and thereby the solution was subjected to the accelerated test. The solution had a Hazen color number (APHA) according to JIS-K3331 of 250.

### Comparative Example 1

Into a reaction container equipped with a condenser, a nitrogen inlet line, and a thermometer, 8.4 parts of isopropyl alcohol, 2.6 parts of N-vinylpyrrolidone, and 2 parts of vinyl acetate were charged together. The atmosphere inside the reaction container was made to be nitrogen atmosphere by introduction of nitrogen under stirring. Then, the reaction container was heated with an oil bath until the inner temperature of the container was 70° C. Thereto, an initiator solution A prepared by dissolving 0.02 parts of t-butylperoxypivalate in 0.84 parts of isopropyl alcohol was added. Then, a monomer solution A including 24 parts of N-vinylpyrrolidone, 18 parts of vinyl acetate, and 33 parts of isopropyl alcohol, and an initiator solution B prepared by dissolving 0.19 parts of t-butylperoxypivalate in 2.4 parts of isopropyl alcohol, were each added dropwise over 4 hours. After completion of the dropwise addition, a monomer solution B including 4.6 parts of N-vinylpyrrolidone and 3.9 parts of isopropyl alcohol, and an initiator solution C prepared by dissolving 0.05 parts of t-butylperoxypivalate in 0.59 parts of isopropyl alcohol, were each added dropwise over 1 hour. After completion of the dropwise addition, the polymerization reaction was performed under stirring for 60 minutes while the inner temperature was kept at 70° C. Then, 50 parts of water was added to the reaction solution to forma homogeneous solution. A distillation device was put on the above-mentioned reaction container and the oil bath temperature was raised to 120° C., and then distillation was started. Three hours later when the removed amount reached 50 parts, the inner temperature of the reaction container was lowered to 80° C. by cooling. Then, the distillation device was removed and an initiator solution D prepared by 0.2 parts of t-butylperoxypivalate in 10 parts of isopropyl alcohol was further added dropwise over 4 hours. After completion of the dropwise addition, the polymerization reaction was performed under stirring for 30 minutes while the inner temperature was kept at 80° C. Then, 80 parts of water was added to the obtained reaction solution to form a homogeneous solution. Then, the distillation device was put on the above-mentioned reaction container again and the oil bath temperature was raised to 120° C., and then distillation was started. Three hours later when the removed amount reached 80 parts, the reaction solution was cooled. As a result, a 53.0% solution of a vinylpyrrolidone/vinyl acetate copolymer was obtained. The N-vinylpyrrolidone concentration was 10 ppm or less and the vinyl acetate concentration was 50 ppm or less.

The obtained polymer solution had a K value of 22. A 50% solution of the copolymer had a Hazen color number (APHA) according to JIS-K3331 of 150, and a 5% solution of the copolymer had an NTU value according to JIS-K0400-9-10 of 3.3. Further, a 50% aqueous solution of the copolymer was prepared from the obtained polymer solution. The 50% aqueous solution 10 g was charged into a 20 mL cylindrical glass container with an inner diameter of 24 mm and a depth of 50 mm, and the container was sealed. The container was kept inside a thermostatic device at 80° C. for 72 hours, and thereby the solution was subjected to the accelerated test. The solution had a Hazen color number (APHA) according to JIS-K3331 of 600.

### Comparative Example 2

Polymerization reaction and distillation were performed in the same manner as in Example 1, except that the time for dropwise addition of the monomer solution A and the initiator solution B was changed from 4 hours to 5 hours and after completion of the dropwise addition of the initiator solution C, the polymerization reaction was performed under stirring for 60 minutes while the inner temperature was kept at 70° C. As a result, a 50.1% white turbid solution of a vinylpyrrolidone/vinylacetate copolymer was obtained. In the distillation, the residual N-vinylpyrrolidone/residual vinyl acetate (ratio by weight) was 0.6/10 when the solvent started to be removed from the reaction solution. At completion of the distillation (at the time of cooling of the reaction solution), the N-vinylpyrrolidone concentration in the reaction solution reduced from 592 ppm that was a concentration before distillation to 10 ppm or less and the vinyl acetate concentration in the reaction solution reduced from 9136 ppm that was a concentration before distillation to 50 ppm or less. The isopropyl alcohol accounted for 0.1% or less.

The obtained polymer solution had a K value of 26, and a 5% solution of the polymer had an NTU value according to JIS-K0400-9-10 of 16.0.

### Comparative Example 3

Polymerization reaction and distillation were performed in the same manner as in Example 1, except that a mixed solvent having a ratio (ratio by weight) of water/isopropyl alcohol of 7/3 was used instead of the mixed solvent having a ratio (ratio by weight) of water/isopropyl alcohol of 5/5. As a result, a 51.6% white turbid solution of a vinylpyrrolidone/vinyl acetate copolymer was obtained. In the distillation, the residual N-vinylpyrrolidone/residual vinyl acetate (ratio by weight) was 1.9/10 when the solvent started to be removed from the reaction solution. At completion of the distillation (at the time of cooling of the reaction solution), the N-vinylpyrrolidone concentration in the reaction solution reduced from 580 ppm that was a value before distillation to 10 ppm or less, and the vinyl acetate concentration in the reaction solution reduced from 3092 ppm that was a concentration before distillation to 50 ppm or less. The isopropyl alcohol accounted for 0.1% or less.

The obtained polymer solution had a K value of 28, and a 5% solution of the polymer had an NTU value according to JIS-K0400-9-10 of 6.9.

### INDUSTRIAL APPLICABILITY

The vinylpyrrolidone/vinyl acetate copolymer of the present invention can be preferably used, for example, as a raw material in production of a styling gel, performed at high temperatures.

1. A vinylpyrrolidone/vinyl acetate copolymer having a residual vinylpyrrolidone content of 100 ppm or less relative to a solid content of the copolymer, the copolymer in 5% by weight aqueous solution form having an NTU value of 4.0 or less, and the copolymer in 50% by weight aqueous solution form having a Hazen color number (APHA) according to JIS-K3331 of 350 or less, the Hazen color number being measured by the following accelerated test.
Accelerated test: the aqueous solution 10 g is charged into a 20 mL cylindrical glass container with an inner diameter of 24 mm and a depth of 50 mm, and the container is sealed and kept inside a thermostatic device at 80° C. for 72 hours.

2. A styling gel comprising the vinylpyrrolidone/vinyl acetate copolymer of claim 1.

3. A process for producing the vinylpyrrolidone/vinyl acetate copolymer of claim 1,
wherein polymerization of a monomer component comprising N-vinylpyrrolidone and vinyl acetate is performed in a solvent in the presence of a polymerization initiator, the solvent containing water and an alcohol at a ratio (ratio by weight) of water/alcohol of 1/9 to 6/4, and
the polymerization is terminated when a ratio (ratio by weight) of residual N-vinylpyrrolidone/residual vinyl acetate is 1/10 or more, and
the alcohol is removed by distillation until the alcohol accounts for 10% by weight or less in a solution of the copolymer.
